(19) United States
(12) Patent Application Publication
(10) Pub. No.: US 2026/0284133 A1
(43) Pub. Date: Sep. 24, 2026

(54) COMPOSITION FOR ACCELERATING AND ENHANCING WOUND HEALING

(71) Applicant: Destiny T Wilcox, Sault Saine Marie, MI (US)

(72) Inventor: Destiny T Wilcox, Sault Saine Marie, MI (US)

(21) Appl. No.: 19/088,878

(22) Filed: Mar. 24, 2025

(51) Int. Cl. A61K 36/48; A61K 9/00; A61K 31/19; A61K 31/728; A61K 36/185; A61K 36/73; A61K 36/736; A61K 36/9066

(52) U.S. Cl. CPC A61K 36/48; A61K 9/0014; A61K 31/19; A61K 31/728; A61K 36/185; A61K 36/73; A61K 36/736; A61K 36/9066

(57) ABSTRACT

A wound healing preparation is provided. A topical composition containing Medicago sativa, sodium hyaluronate, Prunus armeniaca (apricot) kernel oil, Prunus amygdalus dulcis oil, Curcuma longa root, Malus sylvestris, and acetic acid is applied to the wound three times a day, followed by a heat treatment. The composition can be applied directly to the wound by direct application or by use of a spray applicator. The application can be applied in a manner that does not introduce further trauma to the wound.

### FIELD OF THE INVENTION

The present invention relates generally to wound healing preparations. More particularly, the invention relates to a wound healing and skin treatment composition that relies on a blend of topically applied and primarily naturally occurring ingredients, the blend not relying on chemical antiseptics.

### BACKGROUND OF THE INVENTION

Wound healing preparations have been used for many years. The preparations are typically used to disinfect and promote healing. The drawback to topical antiseptic solutions is that they do not promote or enhance healing, they merely prevent or reduce infection. Also, topical antiseptic solutions are often of limited use especially for deep wounds which may require oral antibiotics. Even if the topical antiseptic solutions are effective, they must be applied frequently directly to the wound, sometime the mere application of the solution delays healing by actually causing more trauma.

### SUMMARY

As noted above, one of the unique aspects of the present invention are to combine in a topical composition Medicago sativa, sodium hyaluronate, Prunus armeniaca (apricot) kernel oil, Prunus amygdalus dulcis oil, Curcuma longa root, Malus sylvestris, and acetic acid. The formula of the composition can be in a liquid or a high viscosity form, so that the formula can be applied directly to the wound by direct application or by use of a spray applicator. The composition can be applied in a manner that does not introduce further trauma to the wound. The composition can be used to treat other skin ailments or trauma.

### BRIEF DESCRIPTION OF THE DRAWINGS

FIG. 1 shows a chemical burn on a human scalp to be treated by the inventive compound before application thereof.

FIG. 2 shows the wound of FIG. 1 11 days after treatment.

FIG. 3A shows a scalp to be treated for alopecia with the inventive compound.

FIG. 3B shows the scalp of 3A after 6 days of treatment.

FIG. 4 shows a bite wound on a dog to be treated by the inventive compound before application thereof.

FIG. 5 shows the bite wound after a first week of treatment.

FIG. 6 shows the bite wound after a month of treatment.

FIG. 7 shows another bite wound on a dog after stitches and a drainage tube are applied.

FIG. 8 shows the bite wound of FIG. 7

### DETAILED DESCRIPTION

As stated above, novel combinations of ingredients applied using the inventive method can be used to treat wounds on humans and animals. These combinations can be used to create topical skin compositions that promote wound healing. Prior art methods of healing wounds include stitches, antiseptics, surgery, and constant cleaning. The application of stitches, antiseptics, surgery, and constant cleaning has the drawback of causing additional trauma by physical abrasion etc., and also can cause significant pain to the subject. This solution approaches wound care by requiring minimal contact to the wound. In order to effect the minimal contact approach, the ingredients are provided in the form of a low viscosity preparation that can be sprayed or aerosolized onto the wound in the initial phases of treatment, and a relatively high viscosity preparation that can be liberally spread onto and into the wound once partial healing has occurred and the wound is no longer “tender”.

The inventive preparation can also be applied to other skin trauma or ailments. Burns, wrinkles, scar tissue, age spots, etc., can all benefit by application of the composition as will be shown below.

The inventive preparation can be prepared by combining the ingredients as followed for a liquid (sprayable) preparation:
430 mg Medicago sativa leaf,
8 ml sodium hyaluronate,
9 ml Prunus armeniaca oil,
9 ml Prunus amygdalus dulcis oil,
⅛ teaspoon Curcuma longa root,
2 oz Malus sylvestris, 94% water, 5% acetic acid.

A more viscous preparation can be made with the following ingredients:
430 mg Medicago sativa leaf,
8 ml sodium hyaluronate,
9 ml Prunus armeniaca oil,
9 ml Prunus amygdalus dulcis oil,
4 tablespoons of Butyrospermum parkii.

Butyrospermum parkii (shea butter) is a slightly yellowish or ivory colored natural fat extracted from the seed of the African shea tree, Butyrospermum parkii. It can be used as a moisturizer and salve and in the present inventive preparation is used as a base to create a spreadable balm like consistency.

Prunus armeniaca (apricot) kernel oil is an oil from apricot kernels. In some instances, Prunus armeniaca (apricot) kernel oil is commercially available.

Medicago sativa is made from the leaves of the alfalfa plant. Alfalfa leaves are used in traditional medicine to treat a variety of health conditions. They are rich in vitamin K, which is used to stimulate blood clotting. Alfalfa leaves have anti-inflammatory and antioxidant properties.

Sodium hyaluronate is a substance that occurs naturally in the body and is used in many medical and cosmetic applications. It's also known as hyaluronic acid.

Prunus amygdalus dulcis oil, also known as sweet almond oil, is a light, non-scented oil extracted from almond seeds. The oil is rich in fatty acids and vitamin E, which help the skin retain moisture and protect it from environmental stressors and contains antioxidants.

Curcuma longa root, also known as turmeric, is a plant that has been used in traditional medicine to treat a variety of conditions. Turmeric contains curcumin, a chemical that may help reduce inflammation and swelling.

Malus sylvestris, also known as the European crab apple, has been used for a variety of medicinal purposes. The crushed fruit can be used as a poultice to treat small wounds and inflammation.

The extracts described herein can be extracts made through extraction methods known in the art and combinations thereof. Non-limiting examples of extraction methods include the use of liquid-liquid extraction, solid phase extraction, aqueous extraction, ethyl acetate, alcohol, acetone, oil, supercritical carbon dioxide, heat, pressure, pressure drop extraction, ultrasonic extraction, etc. Extracts can be a liquid, solid, dried liquid, re-suspended solid, etc.

Once the ingredients are placed in a vessel, they can be mixed as by stirring or shaking until a uniform liquid or paste (with a balm like consistency) is formed. The composition is now ready for use.

As mentioned above, conventional methods for treating “serious” or deep wounds involve repeated cleaning and application of antiseptics which can induce additional trauma to the wound area and actually slow or prevent healing, especially when the antiseptics are ineffective. The present method allows for treating the wound with minimal to no physical contact, the only touching (initially) being the droplets of the preparation sprayed onto the wound.

Application of the preparation is as follows. After initial cleaning and inspection of the wound the preparation is sprayed onto the wound using a container with a spray nozzle in the well known manner. Alternatively, the preparation may be massaged into the wound if the preparation is prepared using Butyrospermum parkii to create a spreadable consistency. After spraying a liberal amount of the preparation onto the wound, dry heat is applied for at least 30 minutes. The dry heat may be supplied by a heat gun or equivalent device. A heating pad or equivalent can also be used as would be apparent to one of skill in the art. The application and heating process are performed 3 times per day. When the wound is partially healed, the balm like preparation may then be rubbed onto and into the wound and also applied 3 times a day, with the heating applied as above.

In a key aspect of the invention, the preparation is applied in liquid form initially to avoid physically touching the wound. Once the wound is partially healed, that is, no longer painful or susceptible to infection, the preparation with the shea butter base may be massaged into the wound, which works the preparation deeper into the tissue and speeds healing.

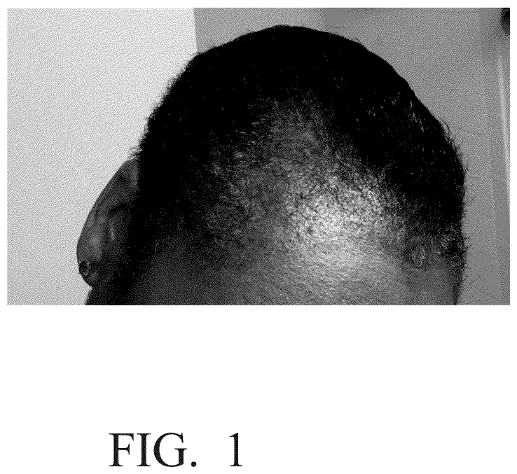

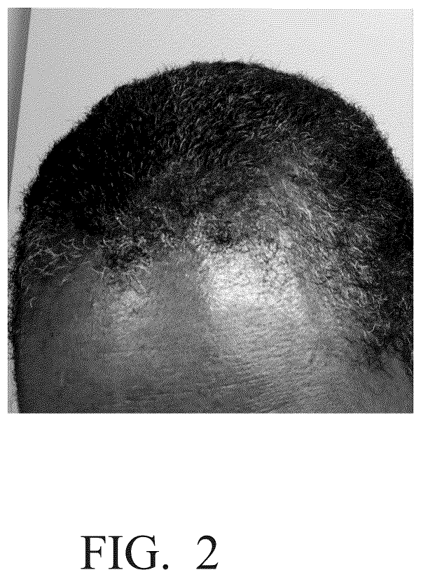

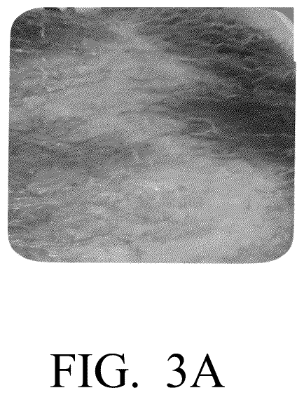

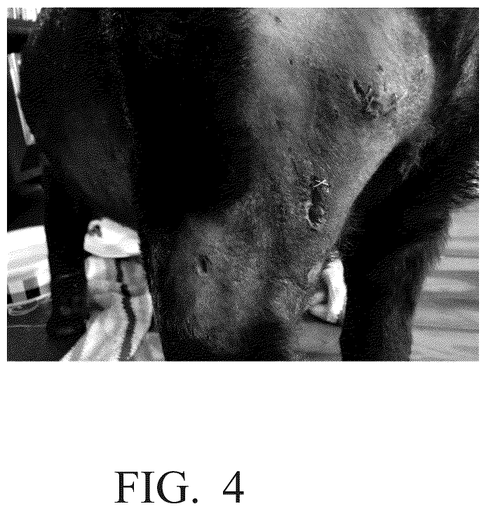

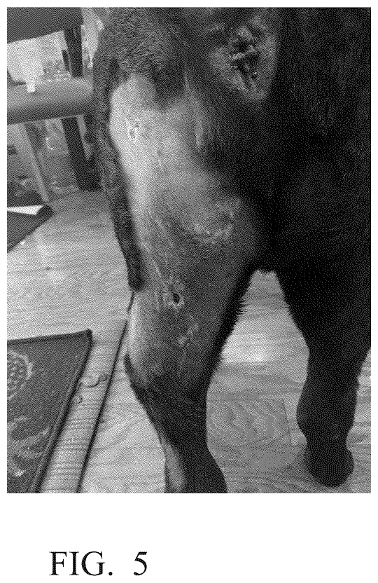

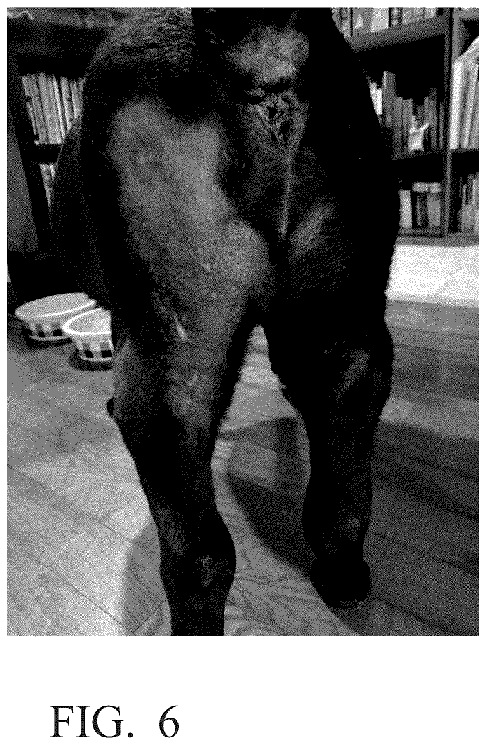

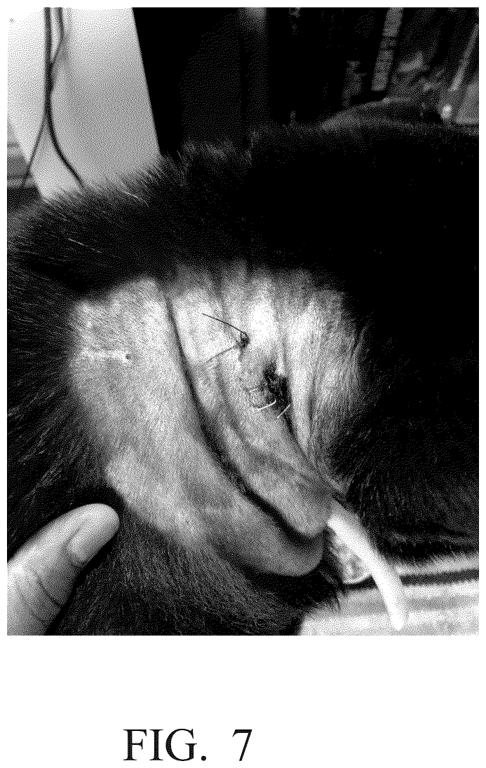

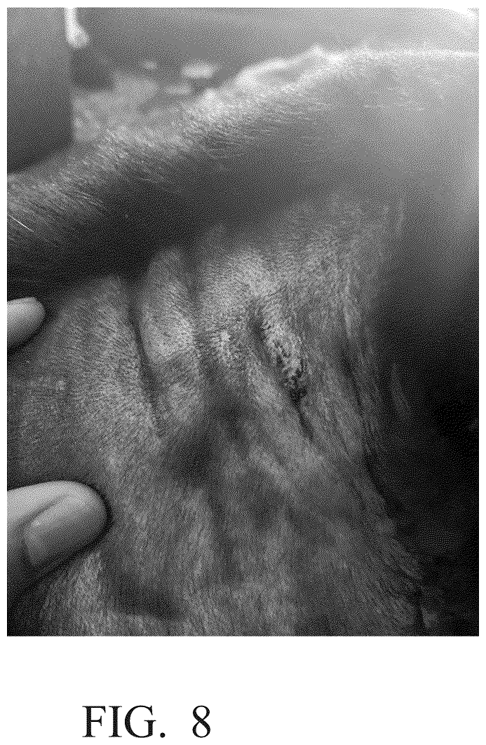

Referring now to FIGS. 1-8 , human and animal wounds/conditions treated by the inventive preparation are shown. In FIGS. 1 and 2 the wounds are chemical burn wounds on a human scalp. The photo in FIG. 2 shows the wounds after 11 days of treatment. FIG. 3 shows a scalp treated for alopecia. The method of the invention is effective to regrow or repair skin in the scalp region which is affected by alopecia. FIG. 3 shows the scalp after an 11 day treatment regimen done in accordance with the above described method. FIGS. 4-6 show bite wounds on a dogs hindquarters. In FIG. 4, the wound is still open and not healed, this is before the application of the preparation. In FIG. 5, an initial phase of healing is shown after 7 days. In FIG. 6, a second phase of healing is shown after another 8 days, the wound nearly healed and safely past the threat of infection. In FIG. 7, an initial phase of healing of a second canine wound is shown. In FIG. 8, the second wound is shown nearly healed after 8 days of treatment. In FIG. 6, a wound healed using the preparation is shown. The inventive method not only promotes and accelerates healing, it does so in a relatively pain free way, as the wound is not touched until at least partially healed. The composition and method of the invention can be used to speed the healing of burns and abrasions as well.

1. A method of treating a wound, the method comprising topically applying to the skin a composition comprising Medicago sativa, sodium hyaluronate, Prunus armeniaca (apricot) kernel oil, Prunus amygdalus dulcis oil, Curcuma longa root, Malus sylvestris, and acetic acid wherein the skin is treated.

2. The method of claim 1 wherein said composition includes water and is applied to said wound in aerosolized form.

3. The method of claim 1 wherein said composition includes Butyrospermum parkii.

4. The method of claim 1 wherein after application of said composition, heat is applied for a period of 30 minutes.

5. The method of claim 4 wherein said composition and heat application are applied 3 times daily.

6. The method of claim 1 wherein said composition is applied in aerosolized form until partial healing of said wound occurs.

7. The method of claim 3 wherein said composition is massaged into the wound.
